(19) United States
(12) Patent Application Publication
(10) Pub. No.: US 2015/0084252 A1
(43) Pub. Date: Mar. 26, 2015

(54) Medicinal Expanded Vile Holder with Sharps Injury Preventer and Storage System

(71) Applicant: Brent Alan Dallman, Indianapolis, IN (US)

(72) Inventor: Brent Alan Dallman, Indianapolis, IN (US)

(21) Appl. No.: 14/033,911

(22) Filed: Sep. 23, 2013

(51) Int. Cl. A61J 1/16; A61B 19/02

(52) U.S. Cl. CPC A61J 1/16; A61B 19/0256; A61B 19/0288
USPC 269/5; 206/366; 604/403

(57) ABSTRACT

A jacket retains a cylindrical drug vial and includes a substantially planar base having a length and a width, the length being greater than the width. A throughhole is sized to receive at least the lid of the vial therethrough. Two ramped devices project from a bottom surface of the base and are substantially aligned along the length of the base. The ramped devices are disposed on opposite sides of the throughhole.

### BACKGROUND

The present invention relates to sharps injury protection, drug labeling, drug storage and drug dispensing systems.

In healthcare facilities, such as hospitals, drugs are administered to patients. Such drugs are typically stored in a cabinet or closet and are categorized by drug and dosage. Some, more advanced, facilities utilize an automated drug inventory system which uses a user interfaced processor to track the drugs being stocked in and taken from the drug cabinet. Some processors employ mechanisms to restrict access to certain drugs. Despite these drug inventory systems, such drug cabinets are still stocked by humans capable of error. Furthermore, highly concentrated injectable pharmaceuticals are often among the medications stored. Highly concentrated pharmaceuticals are often contained in small vials with a capacity as little as 1 milliliter up to 10 milliliters. The small sized vial limits the amount of information these products can display. Consequently, lettering, numbering and electronic coding are competing for this valuable space witch restricts the amount of information that can be conveyed. Compounding this problem of limited labeling area is the possibility of increased labeling needs in the future.

There have been numerous documented cases of drug cabinets being improperly stocked and unintended outcomes resulted. This may be more prone to occurring in emergency settings when caregivers must act quickly. For instance, the healthcare provider may reach into the drug cabinet and remove a vial of medication in a concentration appropriate for a child. Unfortunately, the vial may actually be the adult concentration that was improperly stocked. Administered the adult dose to a child may result in unintended outcomes. In other cases, the wrong drug altogether is placed in a drug compartment. Compounding the problem of inadvertent placement of medications in the wrong section of drug cabinets is the difficulty healthcare workers may have reading drug information such as drug name and concentration on very small vials. These electronic codes often have less than the desired amount of information contained in within them due to space constraints. This lack of space limits employing additional time saving conveniences such as constant flow reordering whereby a facility's scanning of medications as they are utilized triggers the relay of the need for more product to be ordered. This type of interface is an integral part of an efficient supply chain saving time and money for the end user.

Another problem is that when a nurse attempts to insert the needle of a syringe through the lid of a vial in order to draw the drug into the syringe, the healthcare worker may accidentally stick one of her fingers holding the vial with the needle resulting in a sharps injury. In addition to the associated pain, the needle, syringe and medication must be discarded to avoid administering a contaminated drug. Prevention of needle sticks then prevents delays and wasted resources ultimately saving time and money.

Yet another problem is that healthcare facilities such as hospitals often need to augment the manufacturer's labeling with institution-specific pharmacy information such as “highly concentrated” or “caution, paralytic agent.” There may also be facility specific storage advisories that need to be affixed to medications such as “keep refrigerated.” This additional labeling is often applied within a facility's pharmacy. In order to accommodate these requirements on such small vials, additional labeling often covers or has the potential to cover if applied haphazardly the manufacturer's original labeling.

A need remains for a system that helps to insure that the proper drug is stocked in the proper compartment and/or that the proper drug is dispensed and administered to the patient. A need also remains for small medication vials to have a greater surface area to to provide for larger lettering and more detailed electronic coding. A need also remains for a way to avoid needle sticks when the drug is being withdrawn from the vial. A need remains for pharmacy personnel to apply facility-specific advisories on products without risk of obscuring manufacturer's labeling.

### SUMMARY

The present invention provides a system for storing and/or dispensing drugs. In one aspect, the present invention provides a drug storage and dispensing system for storing drugs contained in a container. In one embodiment, the storage system includes a storage tray and a drug identification key member. The storage tray includes at least one drug storage receptacle including a keyed pattern of one or more projections and/or projection-receiving recesses. A holder for a drug container includes a complementary pattern of one or more projections and/or projection-receiving recesses that mates with the tray receptacle such that the drug container holder is securely retained by the tray receptacle. The complementary configurations/shapes of the projections and/or projection-receiving recesses correspond to at least one feature of the drug. The one or more projections are received in the one or more recesses when the container is received in the storage tray.

The invention comprises, in one embodiment thereof, a jacket retaining a cylindrical drug vial having a diameter oriented in a horizontal direction. The jacket includes a conically-shaped collar flaring in an upward direction. An upper end of the collar has a diameter at least 5% greater than the diameter of the vial. The collar has a vertically oriented channel sized to receive the vial therein. At least one leg extends downwardly from the collar. The leg has an outwardly facing panel having information printed thereon.

The invention comprises, in another embodiment thereof, a jacket for retaining a cylindrical drug vial. The vial has a diameter oriented in a horizontal direction. The jacket includes a collar having an upwardly facing surface. The collar has a width at least 5% greater than the diameter of the vial. The collar has an opening sized to receive the vial therein. A body is attached to a bottom of the collar and has a vertically oriented channel in communication with the collar opening. The channel is sized to receive the vial therein. The body includes an opening through which the vial is visible when the vial is in the channel.

The invention comprises, in yet another embodiment thereof, a drug vial storage system including a jacket having a collar with a vertically oriented opening sized to receive the vial therein. At least one leg is connected to the collar. The leg has an outwardly facing panel with information printed thereon. A bottom end of the leg includes at least one slot therein. A location of the slot along the lower end is dependent upon contents of the vial to be retained by the jacket. A tray has a plurality of sets of recesses therein. At least one of the sets of recesses matingly receives the lower end of the leg such that a portion of the tray between the recesses is received in the slot of the leg. Locations of the recesses in the tray are dependent upon contents of the vial to be retained by the jacket.

An advantage of the invention is that it may provide large surface areas on the jacket on which drug-identifying information may be printed. Thus, important information printed on the jacket may be viewed quickly without having to turn or manipulate the vial in order to read printing on a circumferential surface of the vial, thereby saving time and improving accuracy.

Another advantage is that the invention may provide improved and less obstructed visualization of the contents of the vial.

Yet another advantage is that the jacket may provide improved ability to maneuver and hold the vial due to the elongated panels.

Still another advantage is that because the need for a label on the vial may be eliminated, the user may more easily view the contents of the vial through a gap or window in the jacket. Thus, the user may be able to see contamination, debris, bacteria, mold, etc. in the drug.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is an exploded perspective view of a medicinal vial assembly in accordance with one embodiment of the present invention;

FIG. 2a is a perspective view of another embodiment of a medicinal vial assembly of the present invention;

FIG. 2b is another perspective view the medicinal vial assembly of FIG. 2a;

FIG. 3 is a perspective view of the medicinal vial assembly of FIG. 2a with a user inserting a needle of a syringe therein;

FIG. 4 is a side, partially cross-sectional view of yet another embodiment of a medicinal vial assembly of the present invention;

FIG. 5 is a perspective side view of still another embodiment of a medicinal vial assembly of the present invention;

FIG. 6 is a perspective front view of a further embodiment of a medicinal vial assembly of the present invention;

FIG. 7 is a perspective view of yet another embodiment of a medicinal vial assembly of the present invention;

FIG. 8 is a perspective view of an indexing and storage system of the present invention;

FIG. 9 is an enlarged fragmentary, exploded view of the tray and a jacket of a medicinal vial assembly of FIG. 8;

FIG. 10 is a perspective view of still another embodiment of a medicinal vial assembly of the present invention;

FIG. 11 is a front view of a further embodiment of a medicinal vial assembly of the present invention;

FIG. 12 is a top view of the medicinal vial assembly of FIG. 11;

FIG. 13 is a perspective view of yet another embodiment of a medicinal vial assembly of the present invention;

FIG. 14 is a front sectional view of the medicinal vial assembly of FIG. 13 along line 14-14 with the cap removed;

FIG. 15 is a top view of the medicinal vial assembly of FIG. 13 with the cap removed;

FIG. 16 is a perspective view of a further embodiment of a medicinal vial assembly of the present invention;

FIG. 17 is a front view of the medicinal vial assembly of FIG. 16;

FIG. 18 is an exploded front view of the cap and adjustable clamp of the medicinal vial assembly of FIG. 16;

FIG. 19 is an exploded front view of the medicinal vial assembly of FIG. 16; and

FIG. 20 is a perspective view of the medicinal vial assembly of FIG. 16 with the cap open and being held by a user.

Corresponding reference characters indicate corresponding parts throughout the several views. Although the drawings represent embodiments of the present invention, the drawings are not necessarily to scale and certain features may be exaggerated in order to better illustrate and explain the present invention. Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

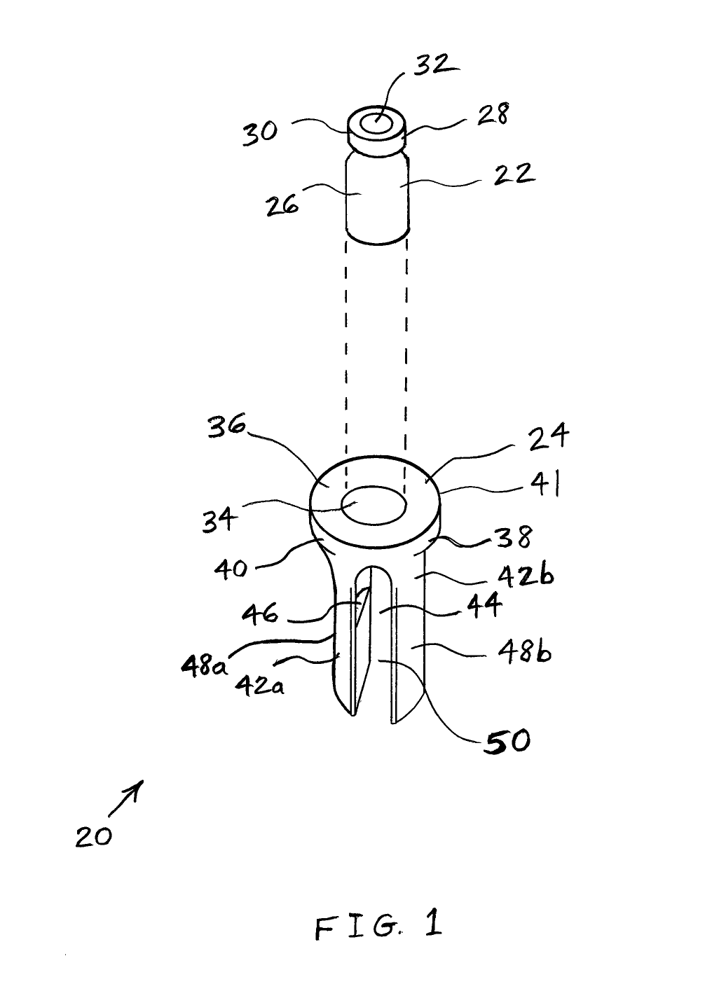

Referring first to FIG. 1, a medicinal vial assembly 20 in accordance with one embodiment of the present invention is described hereinbelow. Medicinal vial assembly 20 generally includes a medicinal vial 22 and a vial jacket 24. Vial 22 may be substantially cylindrically shaped. Vial 22 may have a transparent glass body 26 with an upper circular mouth or opening that is covered by a lid 28. Lid 28 includes a metal annular outer portion 30 which engages body 26, and a circular diaphragm 32 which may be pierced by a needle of a syringe (not shown) in order to access a pharmaceutical product that is disposed in body 26.

Jacket 24 may include an upper conically-shaped collar 38 having a circular top surface 36 with a circular throughhole 34 therein. A radially outward surface 40 of collar 38 may flare radially outwardly as top surface 36 is approached. An upper end 41 of collar 38 may have a diameter at least 5% greater than the diameter of vial 22.

Extending downwardly from collar 38 may be two parallel legs 42a-b defining a cylindrical channel 44 there between that is in communication with throughhole 34. Channel 44 and throughhole 34 may each have a diameter that approximately matches a diameter of vial 22 such that vial 22 may be snugly received and retained within channel 44 after being inserted through throughhole 34. Adhesive may be added to an outer surface of body 26 and/or to an inner surface of channel 44 in order to adhere vial 22 to legs 42a-b.

A detent in the form of a shoulder or ledge 46 may be provided on an inner surface of leg 42a and/or leg 42b in order to block and prevent vial 22 from progressing farther down channel 44. Thus, diaphragm 32 may remain easily accessible through throughhole 34.

Each of legs 42a-b may include a respective flat panel surface area 48a-b on which pertinent information may be printed. Panel 48a may be used by the hospital to identify the drug, volume and/or concentration level in vial 22. Panel 48b may be used by the dispensing pharmacy for in-house labeling such as the expiration date, caution paralytic agent, re-order, instructions such as “keep refrigerated”, information such as “highly concentrated”, etc. Such an ability to customize labeling by the dispensing pharmacy may assist providers in managing inventory and improving patient safety. For example, some combination of standard alphanumeric printing, writing, and codes (e.g., QR codes or bar codes) may be placed on panels 48-a-b. A distance between panels 48a-b is less than a diameter of top surface 36.

A gap 50 between legs 42a-b may be approximately between 0.5 cm and 1.5 cm wide. A health care worker may view the pharmaceutical product in vial 22, as well as a needle placed therein, through transparent body 26 and gap 50.

During use, a nurse may hold legs 42a-b between the thumb and finger of one hand. The nurse then may use her other hand to insert the needle of a syringe through diaphragm 32. In the event that the needle misses diaphragm 32, top surface 36 of collar 38 shields and protects the nurse's fingers from the needle and thereby prevents the fingers from being stuck with the needle.

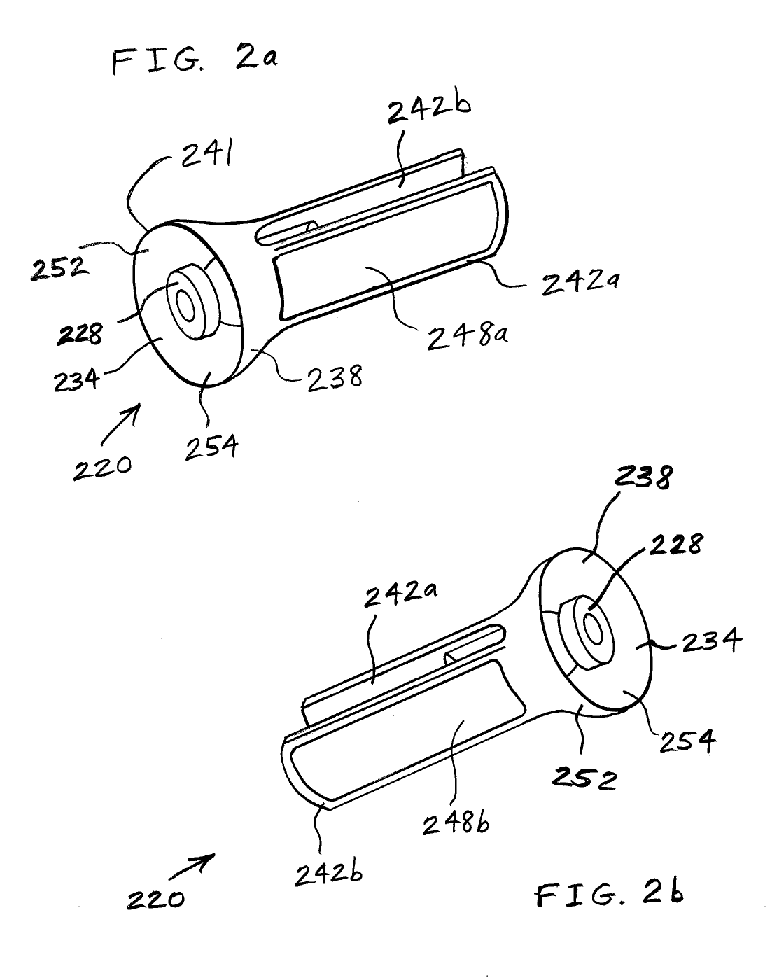

Another embodiment of a medicinal vial assembly 220 in accordance with the present invention is illustrated in FIGS. 2a-b. Medicinal vial assembly 220 includes an upper conically-shaped collar 238 having an annular thin wall 252 defining a mouth or opening 234 at an upper end 241 of collar 238 with a diameter greater than a distance between flat panels 248a-b of legs 242a-b, respectively. An inner diameter of mouth 234 at upper end 241 may be at least 5% greater than the diameter of vial 22. An inner surface 254 of wall 252 may be funnel-shaped and generally upwardly facing, although the direction in which surface 254 faces may also have a radially inwardly facing component.

Assembly 220 may be particularly advantageous in that if the needle misses lid 228, then the needle will very likely hit inner surface 254 and slide down surface 254 with very little danger of sticking a user's finger. The needle in effect may be funneled downward along surface 254 and away from the user's fingers.

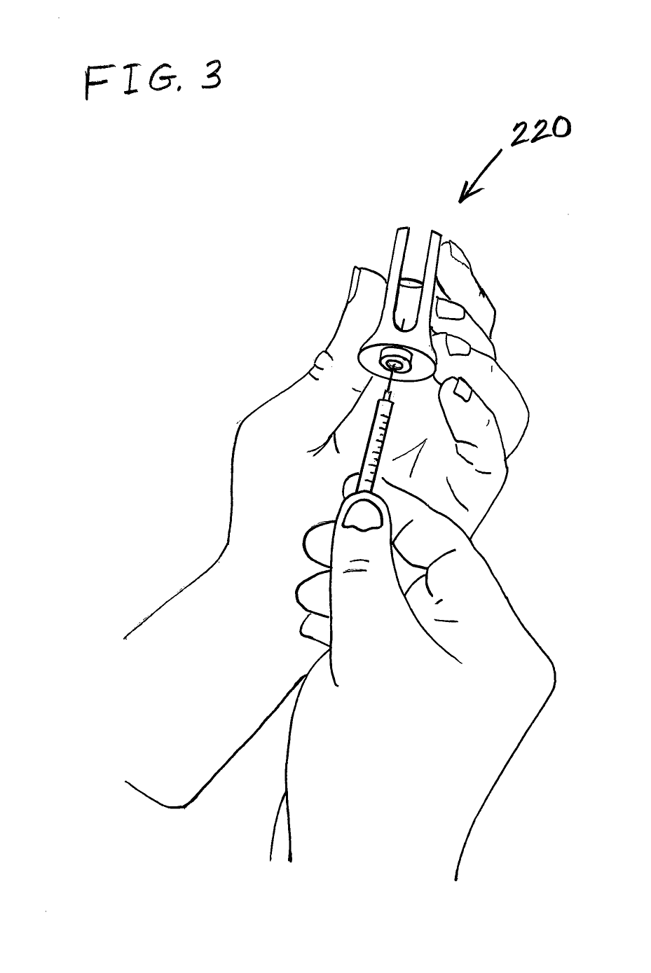

FIG. 3 illustrates assembly 220 in use with a nurse's thumb on one of legs 242a-b and the nurse's fingers on the other of legs 242a-b. The other hand of the nurse is used to insert a needle of a syringe through the lid of the vial. Other features of assembly 220 are substantially similar to those of assembly 20, and are not described herein in order to avoid needless repetition.

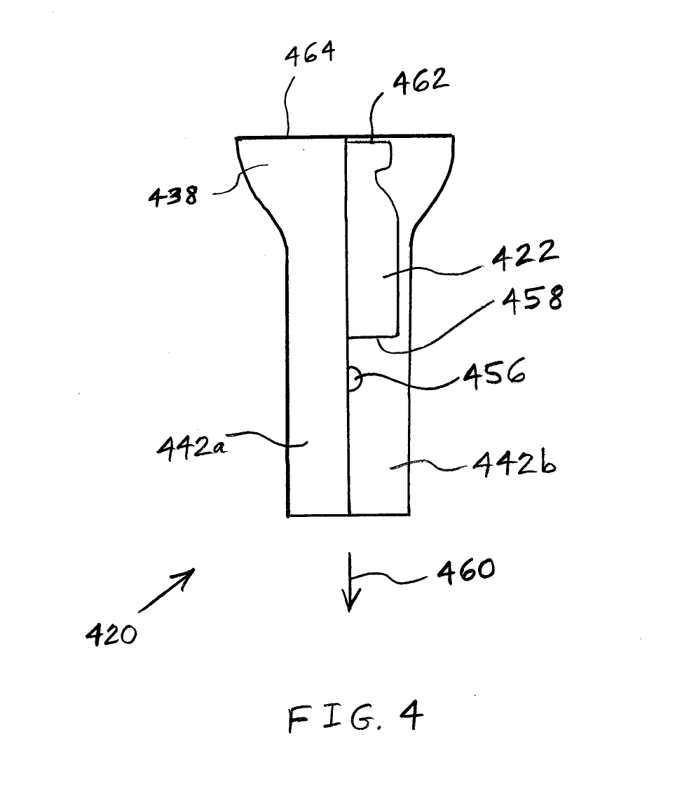

Another embodiment of a medicinal vial assembly 420 in accordance with the present invention is illustrated in the partially cross-sectional view of FIG. 4. Medicinal vial assembly 420 includes a cylindrical detent 456 interconnecting legs 442a-b. Detent 456 prevents a bottom surface 458 of vial 422 from progressing further than the top of detent 456 in downward direction 460. A top surface 462 of vial 422 is below a top annular edge 464 of conically-shaped collar 438. However, it is to be understood that in any of the embodiments disclosed herein the top surface of the vial may be below, flush with, or above the top surface of the jacket.

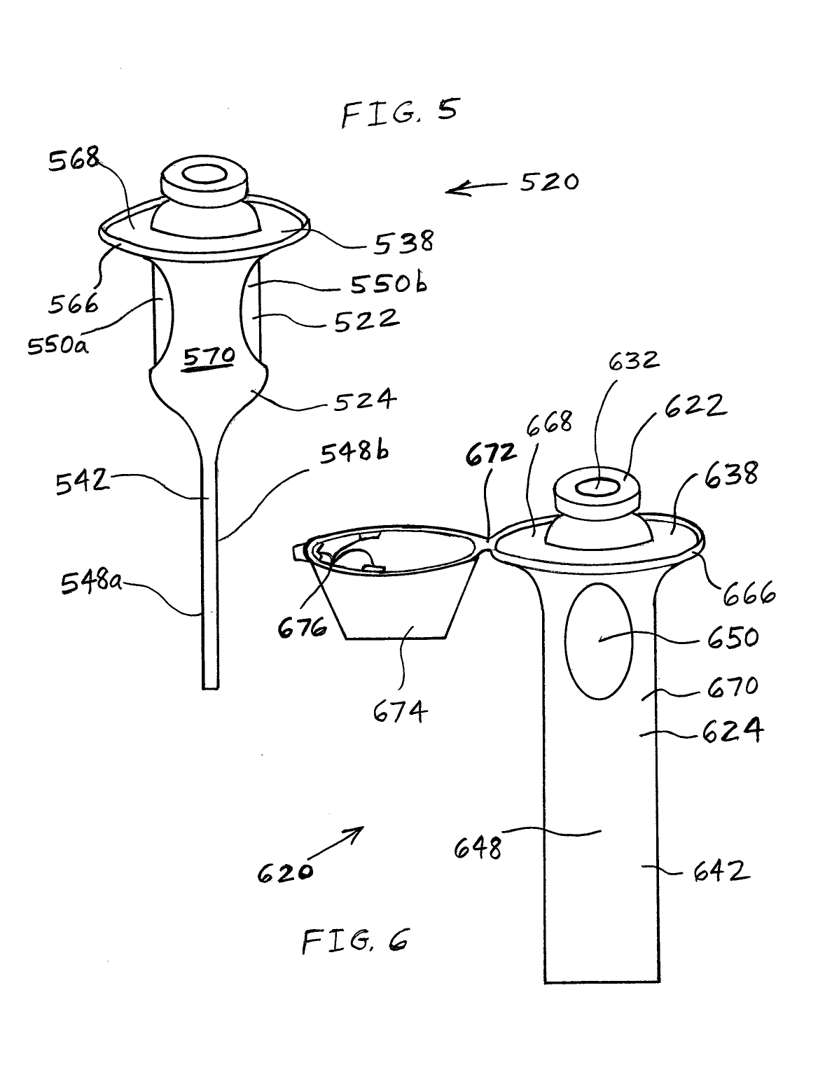

Yet another embodiment of a medicinal vial assembly 520, in accordance with the present invention is illustrated in FIG. 5. Medicinal vial assembly 520 includes a jacket 524 with an annular, horizontally-oriented brim or collar 538 having a ring-shaped upstanding wall 566 at the circumference of collar 538. Wall 566 rises above the level of a top surface 568 of collar 538 such that if a needle point slides in a radially outward direction along top surface 568, the needle may be stopped by wall 566 such that a user's fingers are protected from being stuck by the wayward needle.

Jacket 524 includes a cylindrical body 570 having two opposing oval-shaped windows 550a-b through which the user may view the contents of vial 522. Projecting downwardly from the bottom of body 570 is a planar, rectangular single leg 542 having two flat rectangular printing surfaces 548a-b on opposite sides of leg 542.

Still another embodiment of a medicinal vial assembly 620 in accordance with the present invention is illustrated in FIG. 6. Medicinal vial assembly 620 includes a jacket 624 with an annular collar 638 having a ring-shaped upstanding wall 666 at the circumference of collar 638. Wall 666 rises above the level of a top surface 668 of collar 638 such that if a needle point slides in a radially outward direction along top surface 668, the needle may be stopped by wall 666 such that a user's fingers are protected from being stuck by the wayward needle.

Jacket 624 includes a body 670 having two opposing oval-shaped windows, with only one window 650 being visible in FIG. 6. Through these windows, the user may view the contents of vial 622. Projecting downwardly from the bottom of body 670 are two legs, with only one leg 642 being visible in FIG. 6. Each leg has a flat rectangular outwardly facing printing surface, with only one printing surface 648 being visible in FIG. 6.

Attached to wall 666 via a flexible hinge 672 is a protective cap 674. Cap 674 may be rotated 180 degrees about hinge 672 such that cap 674 covers and protects vial 622. Cap 674 may protect vial 622 from dirt and bacteria, which could particularly cause problems if a diaphragm 632 of vial 622 has already been pierced by a needle. Although cap 674 is not drawn to scale relative to wall 666 in FIG. 6, an inner diameter of cap 674 may be about the same size or slightly larger than an outer diameter of wall 666. Along the inner diameter of cap 674 may be one or more latches 676 which may snap over wall 666 such that cap 674 may be securely fastened in place over wall 666 and vial 622.

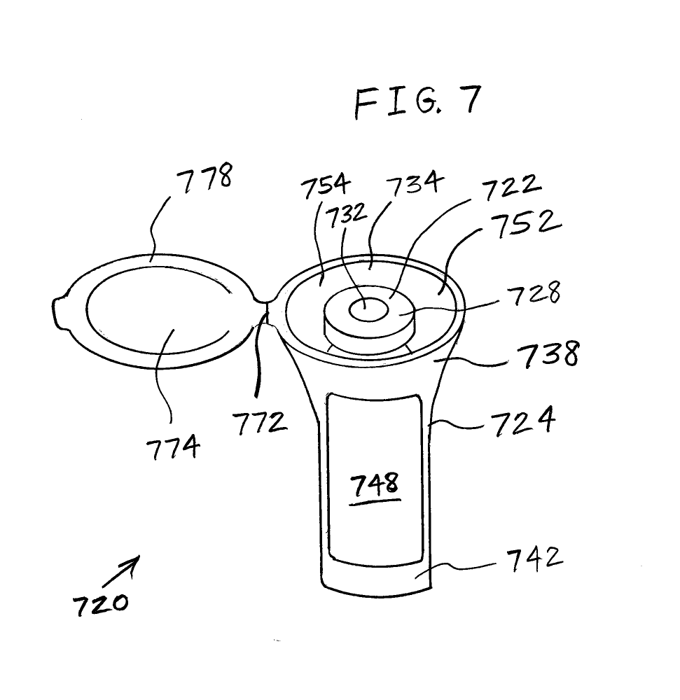

Yet another embodiment of a medicinal vial assembly 720 in accordance with the present invention is illustrated in FIG. 7. Medicinal vial assembly 720 includes a jacket 724 with an upper conically-shaped collar 738 having an annular wall 752 defining a mouth or opening 734 with a diameter greater than a diameter of a lower portion of jacket 724. The lower portion of jacket 724 includes two opposing legs which each have a respective flat panel, although only one leg 742 and one flat panel 748 is visible in FIG. 7. An inner surface 754 of wall 752 may be funnel-shaped.

Like assembly 220, assembly 720 may be particularly advantageous in that if the needle misses lid 728, then the needle will very likely hit inner surface 754 and slide down surface 754 with very little danger of sticking a user's finger. The needle in effect may be funneled downward along surface 754 and away from the user's fingers.

Attached to wall 752 via a flexible hinge 772 is a protective cap 774. Cap 774 may be rotated 180 degrees about hinge 772 such that cap 774 covers and protects vial 722. Cap 774 may protect vial 722 from dirt and bacteria, which could particularly cause problems if a diaphragm 732 of vial 722 has already been pierced by a needle. Cap 774 may include an arcuate groove 778 that is sized to snugly receive the upper edge of wall 752 such that cap 774 may be securely fastened in place over wall 752 and vial 722.

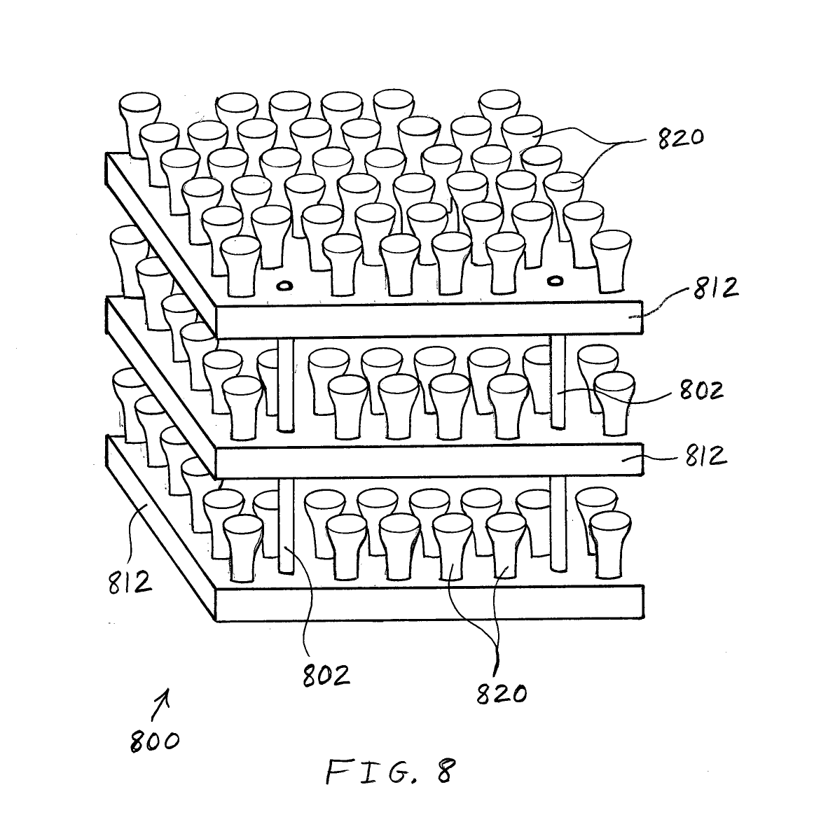

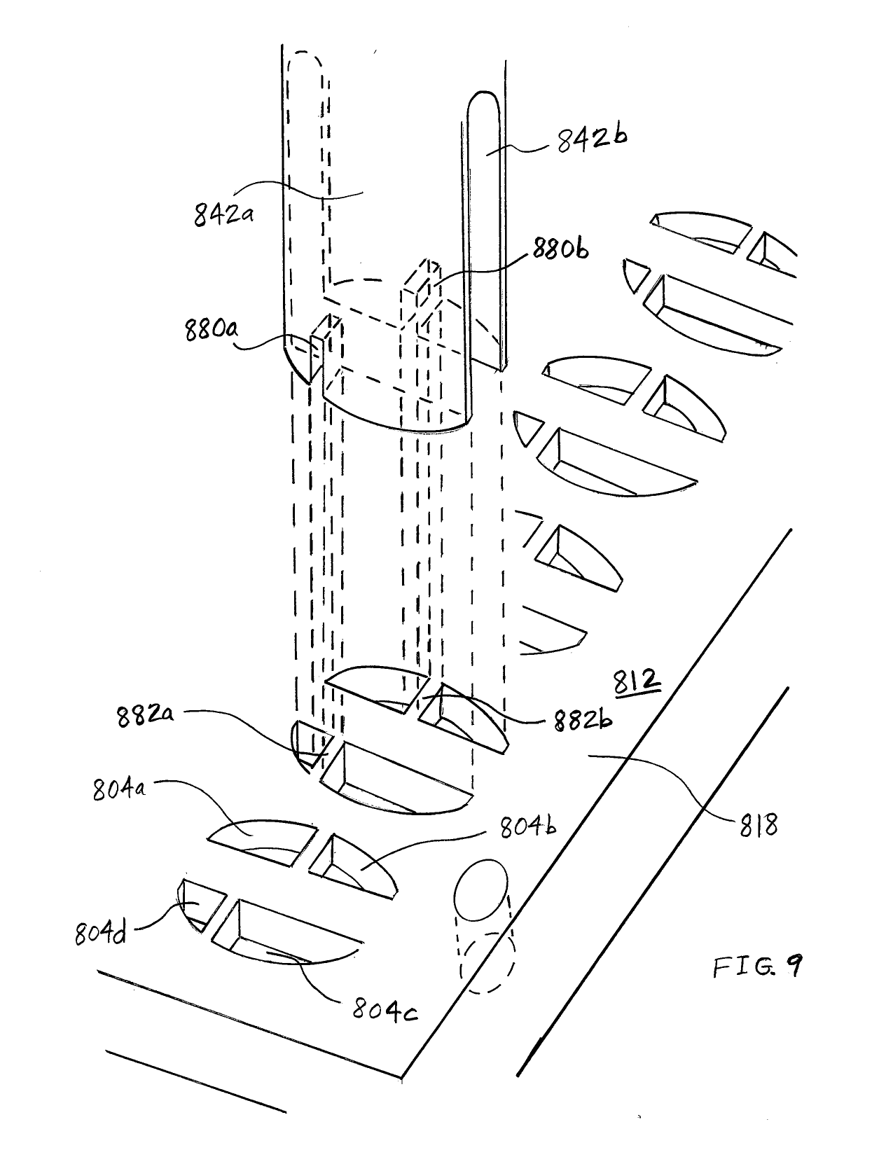

FIGS. 8-9 illustrate an indexing and storage system of the present invention that may be used in conjunction with the medicinal vial assemblies illustrated in FIGS. 1-7. Storage system 800 includes a plurality of storage trays 812 stacked on top of each other by use of four support posts, with the two front support posts 802 being visible in FIG. 8. In the particular embodiment shown in FIG. 8, each tray 812 receives and stores forty-four medicinal vial assemblies 820 of the present invention. As shown in FIG. 9, each storage tray 812 includes a plurality of keyed sets of recesses 804a-d in a top surface 818 of tray 812.

Each vial assembly 820 includes a jacket having two opposing legs 842a-b. Each of legs 842a-b includes a respective slot 880a-b for receiving a respective bridge 882a-b which serves as a barrier between recesses 804a-d. Slots 880a-b and bridges 882a-b have complementary configurations such that each of slots 880a-b is configured to mate with or receive a respective one of bridge 882a-b. The complementary configurations of slots 880a-b and bridges 882a-b are designed to correspond to and identify particular features of the drug to be stored in storage tray 812. For example, the relative sizes of recesses 804a-d and the locations of slots 880a-b on legs 842a-b may identify the drug within each of the vials and its concentration. The panels of legs 842a-b may be entirely above top surface 818 of tray 812 when legs 842a-b are matingly received in tray 812 such that the panels are completely and entirely visible.

In use, storage trays 812 may be placed on storage shelves in drug cabinets, mounted on walls in drug closets, or arranged in any suitable manner in the drug storage area of a healthcare facility. Legs 842a-b and storage trays 812 are equipped with mating drug identification keys that assist in the storage, identification and dispensing of drugs. For instance, complementary configurations of recesses 804a-d and slots 880a-b are designed to correspond to a particular feature of the drug. For example, the locations of bridges 882a-b and slots 880a-b may correspond to a specific drug composition. Accordingly, all jackets containing that specific drug are equipped with legs 842a-b having slots 880a-b, which identifies the drug within the vial. All drug containers containing other drugs are equipped with legs having slots of a different location or shape incapable of being received within recesses 804a-d. Thus, when drug cabinets are stocked, vials cannot be improperly placed in the wrong drug tray due to forced function, thereby, minimizing the chances that the wrong drug will be dispersed.

Storage trays 812 and the jackets may be formed of any rigid material capable of holding vials and forming a mating coupling. For instance, storage trays 812 and the jackets may be formed of rigid plastic, metal, wood and/or combination thereof. Trays 812 and the jackets may be formed by any means including extrusion, molding, machining or other means.

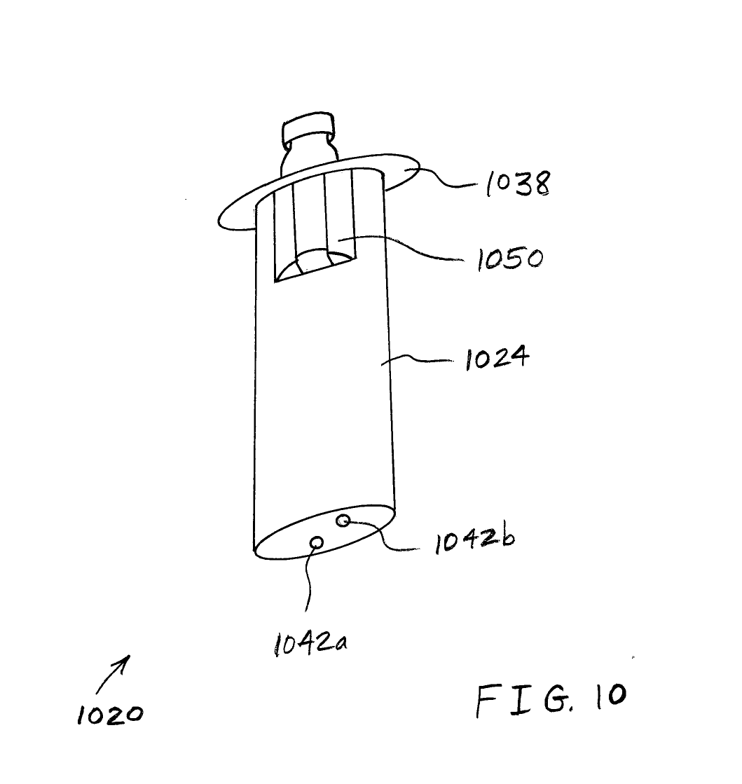

Still another embodiment of a medicinal vial assembly 1020 in accordance with the present invention is illustrated in FIG. 10. Medicinal vial assembly 1020 includes a jacket 1024 with a flat, elliptical shield 1038 having a width greater than a width of jacket 1024. Jacket 1024 includes a transparent window 1050 through which the vial may be viewed. The width of window 1050 may be greater than the width of the vial in order to accommodate and receive the vial therein. A bottom surface of jacket 1024 includes two non-symmetrically positioned holes 1042a-b which may mate with the male projections of a socket of a base (not shown) such that the base supports assembly 1020 in an upright position. The male projections of the base socket may be also non-symmetrically positioned such that the holes and projections function as a key which allows the base to mate with and support only assembly 1020 with its particular hole positioning. Assembly 1020 may receive only a vial of a particular drug having a particular concentration, etc. Thus, assembly 1020 won't mate with the base, the healthcare professional is alerted that the drug in the vial may not be the one that is intended to be supported by the base.

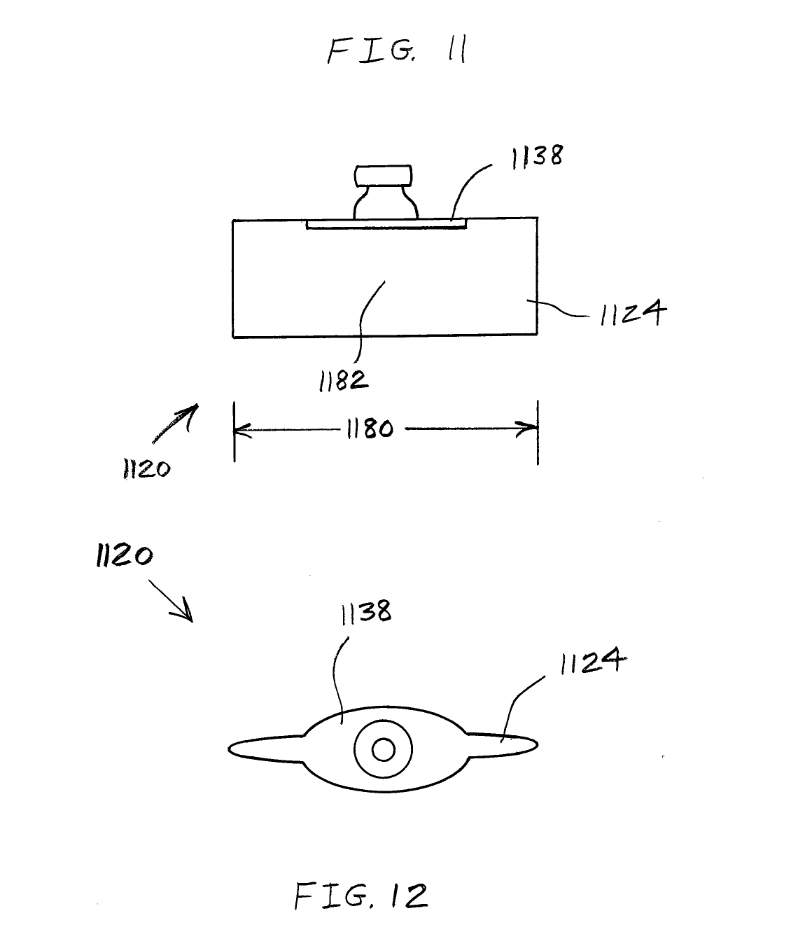

A further embodiment of a medicinal vial assembly 1120 in accordance with the present invention is illustrated in FIGS. 11-12. Medicinal vial assembly 1120 includes a jacket 1124 having a relatively long length 1180 and a flat area 1182 which is large enough to accommodate a large amount of written information. At the top of jacket 1124 is a flat, elliptical shield 1138 having a width greater than a width of jacket 1124. Instead of elliptical, shield 1138 may be other shapes, e.g., circular.

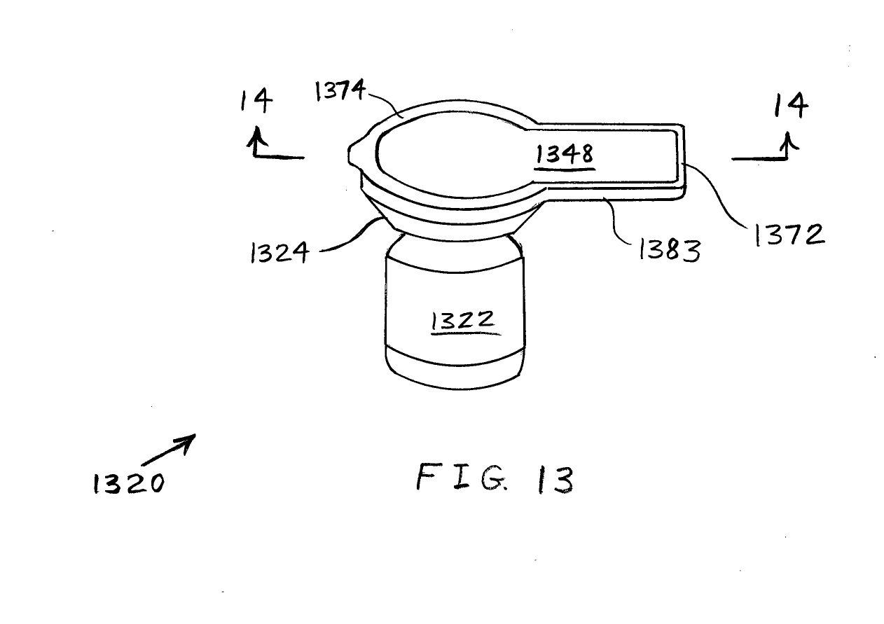

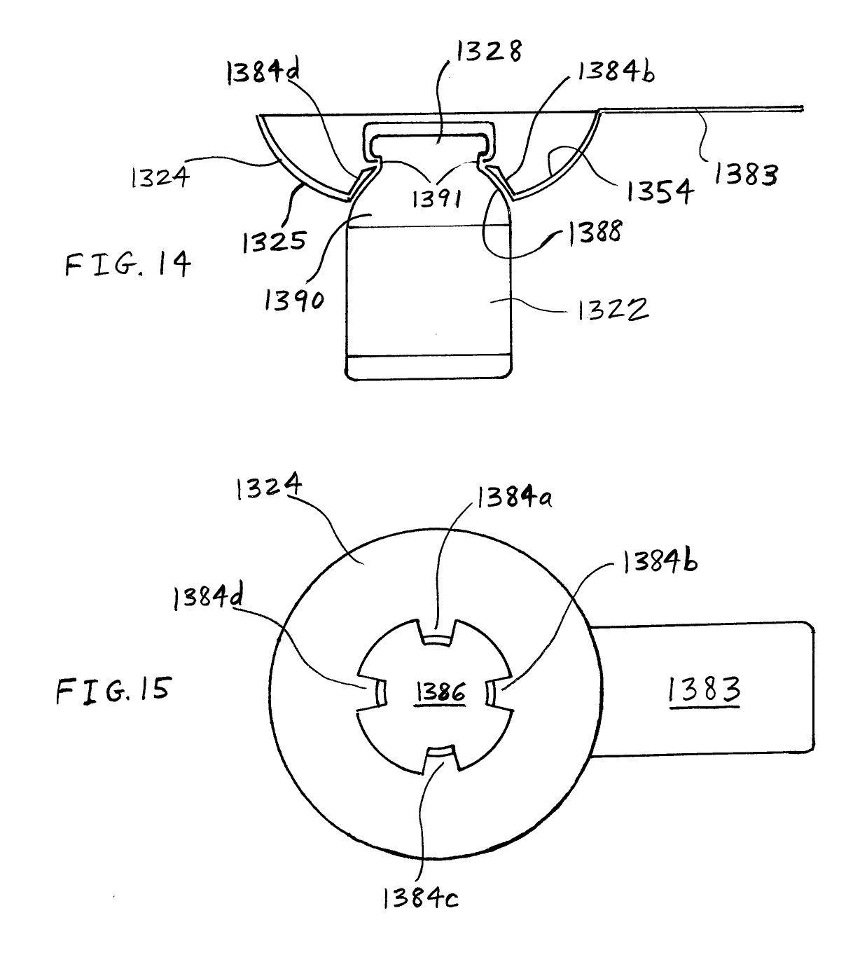

Yet another embodiment of a medicinal vial assembly 1320 in accordance with the present invention is illustrated in FIGS. 13-15. Medicinal vial assembly 1320 includes a jacket 1324 with a bowl-shaped base 1325 and a handle 1383 both sharing a snap-fit cap 1374 with a flat panel 1348 suitable for receiving writing thereon. As shown in FIGS. 14-15, jacket 1324 includes four tabs 1384a-d projecting into a circular throughhole 1386 at an angle of about forty-five degrees relative to the vertical and horizontal directions.

During assembly, vial 1322 may be inserted upwardly through throughhole 1386 until lid 1328 advances past tabs 1384a-d, at which point tabs 1384a-d are clamped between lid 1328 and a slanted outer surface 1388 of a body 1390 of vial 1322. Thus, vial 1322 is effectively attached to jacket 1324. Lid 1328 and surface 1388 each have a diameter that is greater than a diameter of a neck 1391 of vial 1322, which enables tabs 1384a-d to be securely received in neck 1391.

Like assemblies 220 and 720, assembly 1320 may be particularly advantageous in that if the needle misses lid 1328, then the needle will very likely hit an inner surface 1354 of jacket 1324 and slide down surface 1354 with very little danger of sticking a user's finger. The needle in effect may be funneled downward along surface 1354 and away from the user's fingers.

Cap 1374 may be rotated 180 degrees about a hinge 1372 such that cap 1374 covers and protects vial 1322. Cap 1374 may protect vial 1322 from dirt and bacteria, which could particularly cause problems if a diaphragm of vial 1322 has already been pierced by a needle.

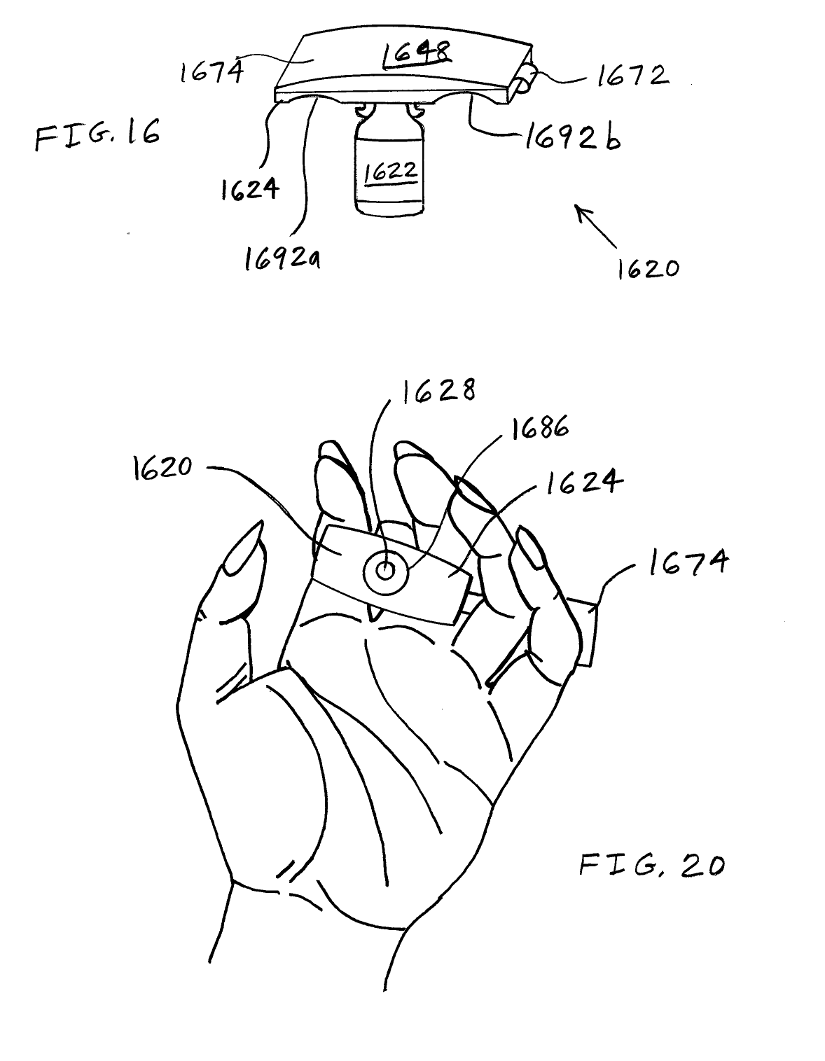

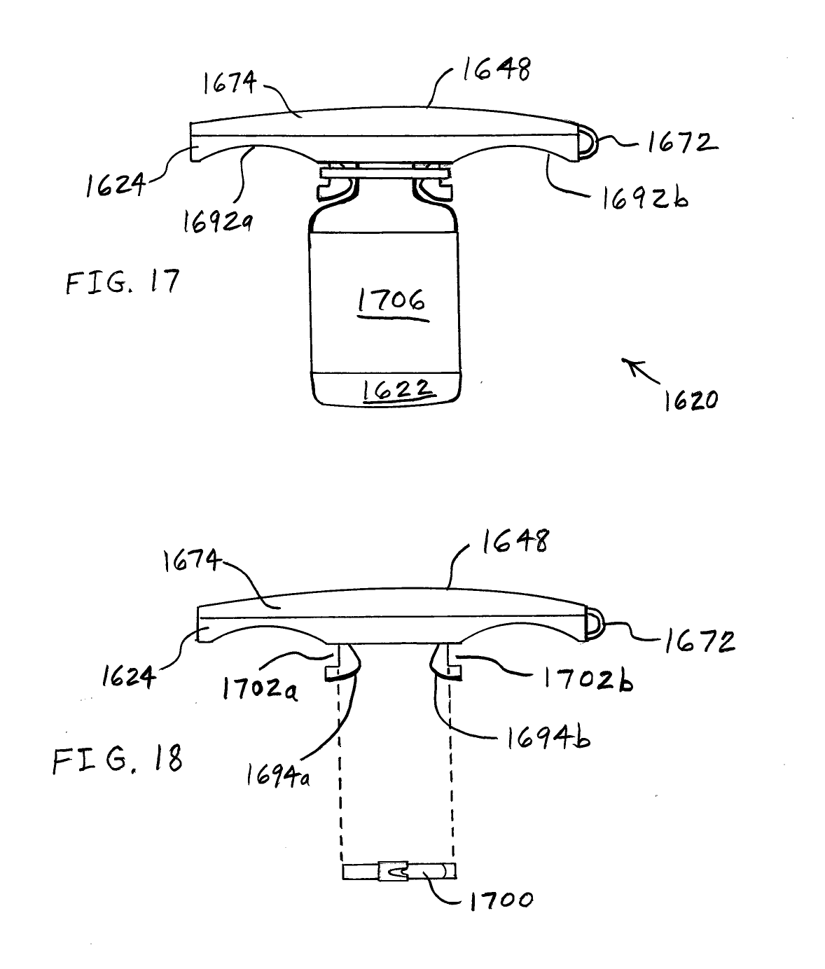

Yet another embodiment of a medicinal vial assembly 1620 in accordance with the present invention is illustrated in FIGS. 16-20. Medicinal vial assembly 1620 includes a substantially flat, rectangular base 1624 having an optional snap-fit cap 1674 with a panel 1648 suitable for receiving writing thereon. The writing may be added by hand or may be on printer foil tags which can accommodate a substantial amount of information. Panel 1648 may also accommodate in-house coding to manage the product once it is received. Panel 1648 can aid a pharmacy in conveying messages to other health care workers, or can carry a message for in-home use. Plastic color coding and/or corporate logos may be employed.

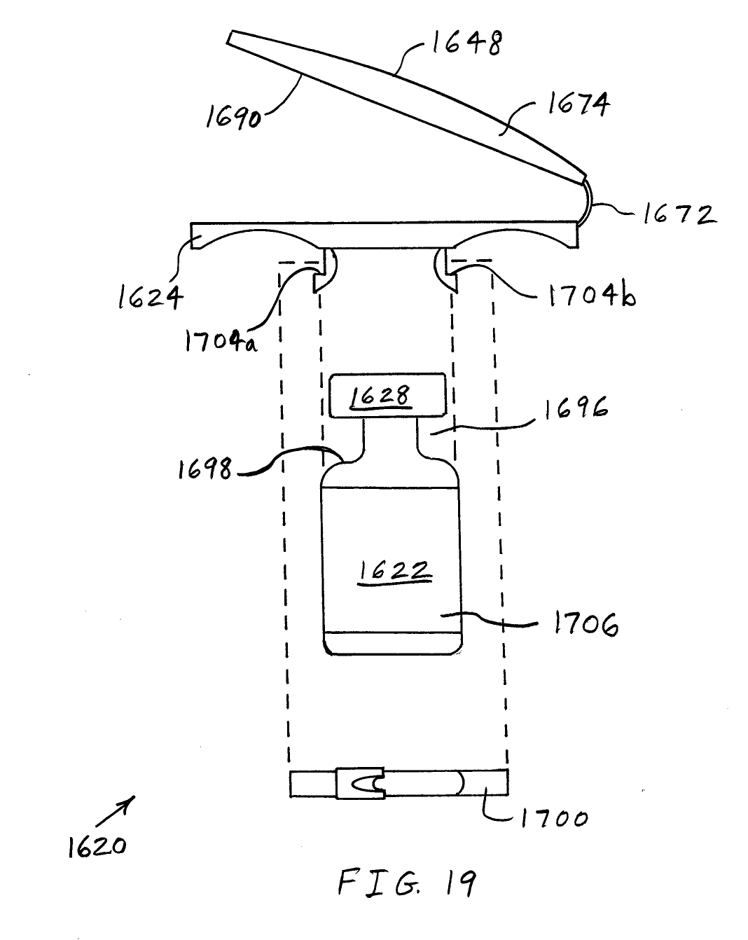

Cap 1674 may be rotated 180 degrees about a hinge 1672, as best shown in FIG. 19, such that cap 1674 covers and protects vial 1622. The connective hinge 1672 may prevent cap 1674 from being lost or dropped, thereby preventing contamination. Cap 1674 may protect vial 1622 from dirt and bacteria, which could particularly cause problems if a diaphragm of vial 1622 has already been pierced by a needle. An underside 1690 of cap 1674 may be employed as a drug packing insert or may carry other valuable information.

Finger grooves 1692a-b may be ergonomically designed to fit the contour of the underside of the caregiver's fingers. Thus, grooves 1692a-b may help create a stable bridge, decreasing the chance of a needle stick (i.e., sharps injury).

During assembly, vial 1622 may be inserted upwardly between two plastic convex ramps 1694a-b which respond by spreading outwardly during the initial insertion of lid 1628. After lid 1628 passes ramps 1694a-b, ramps 1694a-b may spring back inwardly into the recess 1696 between lid 1628 and a shoulder 1698 of vial 1622. After ramps 1694a-b are back in place filling the space between lid 1628 and shoulder 1698, an adjustable clamp 1700 may be placed into cutouts 1702a-b and cinched or tightened such that vial 1622 is effectively attached to base 1624. Clamp 1700 may be a simple zip tie, a wire, or may be made of stainless steel. In the embodiment shown, clamp 1700 is a self-locking tie with a rounded end. A banding tool with cutters may be provided for increased efficiency. Ledges 1704a-b map prevent adjustable clamp 1700 from sliding off of ramps 1694a-b. Both assemblies 1320 and 1620 may have the advantage that an original manufacturer's label, such as label 1706, is fully visible.

As shown in FIG. 20, cap 1674 may be rotated away from base 1624 such that lid 1628 is exposed and assembly 1620 may be gripped such that base 1624 shields the user's fingers from a needle being inserted through lid 1628. Thus, base 1624 may function as a protective barrier for health care workers by reducing the chance of a sharps injury. Base 1624 includes a circular throughhole 1686 through which a user has access to lid 1628.

The systems of the present invention help insure that the correct drug is stocked in the correct location in the drug storage area, thereby minimizing the chances that the wrong drug is selected and administered due to inaccurate stocking. The systems of the present invention also help insure that access to the drugs is selectively granted, thereby minimizing the chances that the wrong drug will be taken from the drug storage area.

In addition to providing a medium for printing or writing, the vial jackets of the present invention may carry electronics, such as plastic-embedded RFID circuit chips, which contain information identifying characteristics of the drug in the vials and allow for more secure shipping. The jacket provides opportunity for RFID chips to be affixed remotely from the liquid of the vial which may cancel the signal.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A jacket for retaining a cylindrical drug vial, the vial having a diameter oriented in a horizontal direction, the jacket comprising:
a collar of variable shape including but not limited to conical, pyramidal, elliptical, flat ring or inverted cone providing a barrier between the needle puncture location on the vial and the body of the vial where the user holds the vial, the collar having a vertically oriented channel sized to receive the vial therein; and
at least one leg extending downwardly from the collar, the leg having an outwardly facing panel configured to have information printed thereon.

2. The jacket of claim 1 wherein the diameter of the collar comprises an outer diameter, the collar including an inner diameter at least 5% greater than the diameter of the vial.

3. The jacket of claim 1 wherein the vial has a transparent body, the jacket having an opening through which contents of the vial are visible.

4. The jacket of claim 1 wherein the collar has an annular top surface with an opening in communication with the channel, the opening and the channel being sized to snugly receive the vial therein.

5. The jacket of claim 1 wherein the outwardly facing panel is substantially flat.

6. The jacket of claim 1 further comprising a cap hingedly attached to an upper end of the collar, the cap being configured to be rotated about the hinge and snap-fit over the upper end of the collar.

7. The jacket of claim 1 wherein the at least one leg includes two opposing legs each having a respective said outwardly facing panel configured to have information printed thereon, the two legs defining a gap there between through which contents of the vial are visible.

8. A jacket for retaining a cylindrical drug vial, the vial having a diameter oriented in a horizontal direction, the jacket comprising:
a collar having an upwardly facing surface, the collar having a width at least 20% greater than the diameter of the vial, the collar having an opening sized to receive the vial therein; and
a body attached to a bottom of the collar and having a vertically oriented channel in communication with the collar opening and sized to receive the vial therein, the body including an opening configured to enable the vial to be visible when the vial is in the channel.

9. The jacket of claim 8 further comprising at least one leg extending downwardly from the body, the leg having an outwardly facing panel configured to have information printed or applied thereon.

10. The jacket of claim 9 wherein the at least one leg comprises a single leg, the outwardly facing panel comprising a first outwardly facing, substantially flat panel, the leg including a second outwardly facing panel, substantially flat panel opposing the first panel.

11. The jacket of claim 8 wherein the upwardly facing surface of the collar is substantially flat.

12. The jacket of claim 8 wherein the upwardly facing surface of the collar is substantially funnel-shaped.

13. The jacket of claim 8 further comprising a cap hingedly attached to the collar, the cap being configured to be rotated about the hinge and snap-fit over the collar.

14. The jacket of claim 8 wherein the leg has a lower end with a slot therein, a location of the slot along the lower end being dependent upon contents of the vial to be retained by the jacket.

15. A drug vial storage system comprising:
a jacket comprising:
a collar having a vertically oriented opening sized to receive the vial therein; and
at least one leg connected to the collar, the leg having an outwardly facing panel configured to have information printed thereon, a bottom end of the leg including at least one slot therein, a location of the slot along the lower end being dependent upon contents of the vial to be retained by the jacket; and
a tray having a plurality of sets of recesses therein, at least one of the sets of recesses matingly receiving the lower end of the leg such that a portion of the tray between the recesses is received in the slot of the leg, locations of the recesses in the tray being dependent upon contents of the vial to be retained by the jacket.

16. The system of claim 15 wherein the panel of the leg has information printed thereon describing contents of the vial to be retained by the jacket.

17. The system of claim 15 wherein both the location of the slot along the lower end of the leg and the locations of the recesses in the tray are indicative of an identity of a drug contained in the vial and a concentration level of the drug.

18. The system of claim 15 wherein the collar has a width at least 0.4 cm greater than the diameter of the vial.

19. The system of claim 18 wherein the leg has a width less than the width of the collar.

20. The system of claim 15 wherein the panel of the leg is entirely above a top surface of the tray when the leg is matingly received in the tray.
